(19) United States
(12) Patent Application Publication
(10) Pub. No.: US 2008/0255672 A1
(43) Pub. Date: Oct. 16, 2008

(54) ORTHOPEDIC IMPLANT

(75) Inventor: Carlos E. Gil, Collierville, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/733,508

(22) Filed: Apr. 10, 2007

(51) Int. Cl. A61F 2/34

(52) U.S. Cl. 623/22.28; 623/22.36

(57) ABSTRACT

An orthopedic implant is disclosed and can include a first component and a second component that can be installed in the first component. The orthopedic implant can further include a seal that can be installed between the first component and the second component. The seal can substantially prevent debris from the second component from migrating through the first component.

### FIELD OF THE DISCLOSURE

The present disclosure relates generally to orthopedic implants. More specifically, the present disclosure relates to ball-and-socket implants.

### BACKGROUND

Osteolytic lesions can be found on arthroplasty implants after several years of implantation. For example, in a patient who has had a hip replacement, osteolytic lesions can occur on the acetabulum. These lesions can be produced by a biological response to ultra-high molecular weight polyethylene (UHMWP) debris. The UHMWP debris can result from wear between an acetabular shell and an UHMWPE liner in stalled therein. Further, the UHMWP debris can migrate through one or more screw holes in the acetabular shell into the interface between the acetabulum and the acetabular shell. Accordingly, there is a need to prevent UHMWP debris from migrating through the acetabular shell of a hip implant.

### BRIEF DESCRIPTION OF THE DRAWINGS

FIG. 1 is a plan view of a hip joint;

FIG. 2 is an exploded plan view of a hip joint;

FIG. 3 is an exploded view of an acetabular implant;

FIG. 4 is a perspective view of an acetabular shell associated with the acetabular implant;

FIG. 5 is another perspective view of the acetabular shell;

FIG. 6 is a cross-section view of the acetabular shell;

FIG. 7 is a detail view of a screw engagement hole associated with the acetabular shell;

FIG. 8 is a perspective view of a seal associated with the acetabular implant;

FIG. 9 is a cross-section view of the seal;

FIG. 10 is a perspective view of an apex plug associated with the acetabular implant;

FIG. 11 is a cross-section view of the apex plug;

FIG. 12 is a perspective view of a liner associated with the acetabular implant;

FIG. 13 is a cross-section view of the liner;

FIG. 14 is a plan view of an assembled acetabular implant;

FIG. 15 is another plan view of the assembled acetabular implant;

FIG. 16 is a bottom plan view of the assembled acetabular implant;

FIG. 17 is a cross-section view of the assembled acetabular implant taken along line 17-17 in FIG. 16;

FIG. 18 is an exploded cross-section view of the acetabular implant; and

FIG. 19 is a flow chart illustrating a method of treating a hip joint.

### DETAILED DESCRIPTION OF THE DRAWINGS

An acetabular implant is disclosed and can include an acetabular shell. Further, a liner can be installed within the acetabular shell. The acetabular implant further includes a seal that can be installed between the liner and the acetabular shell. The seal can substantially prevent debris from the liner from migrating through the acetabular shell.

In another embodiment, a seal for an acetabular implant is disclosed and can include a generally hemi-spherical seal body, wherein the seal body can be placed between an acetabular shell and a liner and wherein the seal body can substantially prevent debris from the liner from migrating through the acetabular shell.

In yet another embodiment, a method of treating a hip joint is disclosed and can include exposing the hip joint. Further, the method can include preparing an acetabulum to receive an acetabular implant and installing an acetabular shell within the acetabulum. Also, the method can include installing a seal within the acetabular shell.

In still another embodiment, an orthopedic implant is disclosed and can include a first component and a second component that can be installed in the first component. The orthopedic implant can further include a seal that can be installed between the first component and the second component. The seal can substantially prevent debris from the second component from migrating through the first component.

In yet still another embodiment, a seal for an orthopedic implant is disclosed and can include a generally hemi-spherical seal body. The seal body can be placed between a first component and a second component. Moreover, the seal body can substantially prevent debris from the second component from migrating through the first component.

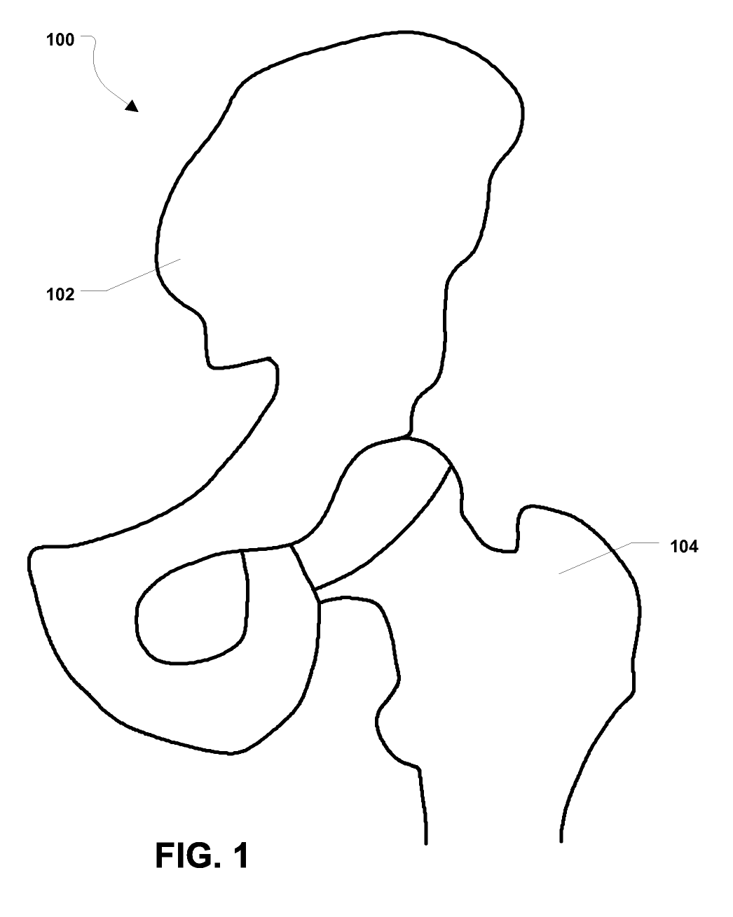

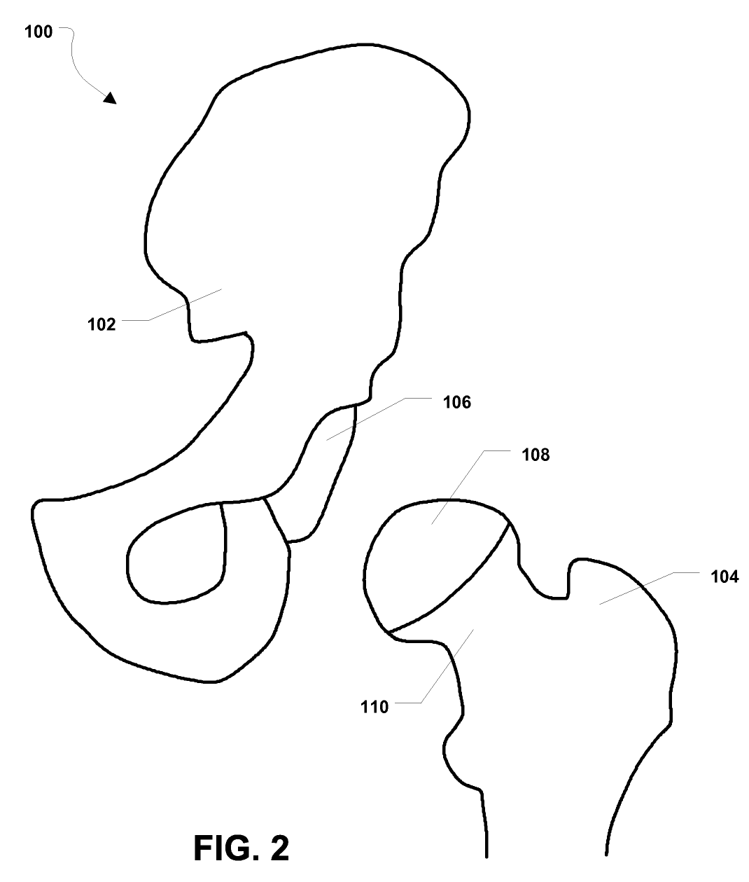

Referring to FIG. 1 and FIG. 2, a hip joint is illustrated and is generally designated 100. As shown, the hip joint 100 can include a pelvis 102 and a femur 104. As shown in FIG. 2, the pelvis 102 can include an acetabulum 106. Further, the femur 104 can include femoral head 108 and femoral neck 110. As depicted in FIG. 1, the femoral head 108 of the femur 104 can fit into the acetabulum 106 of the pelvis 102. Moreover, the femoral head 108 can rotate, or articulate, within the acetabulum 106. Accordingly, the hip joint 100 is a ball-and-socket joint.

As the hip joint 100 ages, the acetabulum 106 or the femoral head 108 can deteriorate and weaken. As such, it may be desirable to replace the hip joint 100 with an artificial hip joint. For example, the acetabulum 106 can be replaced or otherwise augmented with an acetabulum implant, e.g., the acetabulum implant described herein.

### Description of an Acetabular Implant

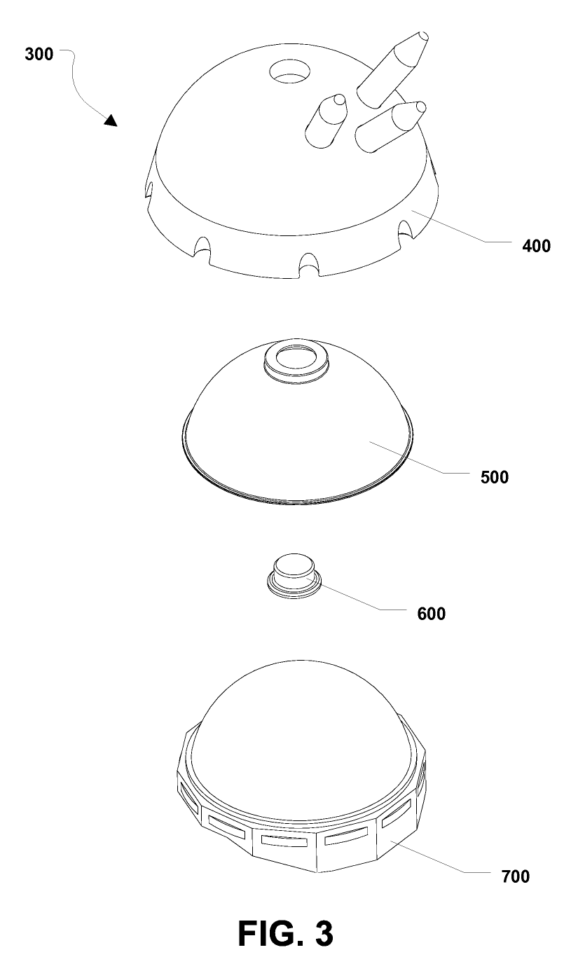

Referring to FIG. 3, an embodiment of an acetabulum implant is shown and is generally designated 300. As shown in FIG. 3, the acetabulum implant 300 can include an acetabular shell 400, a seal 500, an apex plug 600, and a liner 700. In a particular embodiment and as described in greater detail below, the seal 500 can be fitted into the acetabular shell 400 and held in place using the apex plug 600. Further, the liner 700 can be fitted into the seal 600. As described in greater detail below, the seal 600 can prevent debris from the liner 700 from migrating through the acetabular shell 400. As such, the seal 600 can substantially reduce the occurrence of osteolytic lesions around the acetabular shell 400 during the implanted life of the acetabulum implant 300.

### Description of the Acetabular Shell

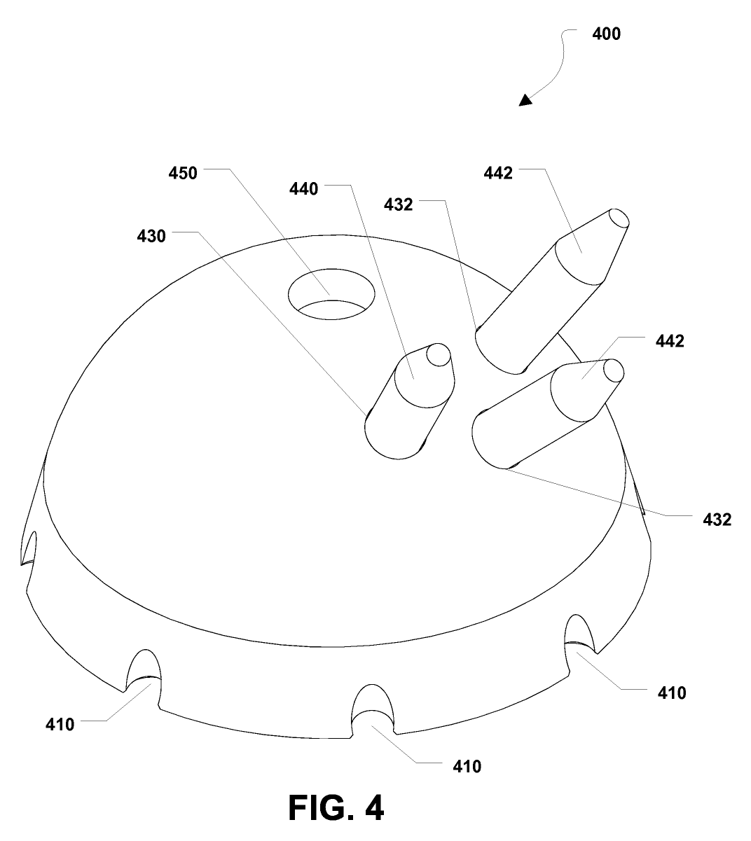

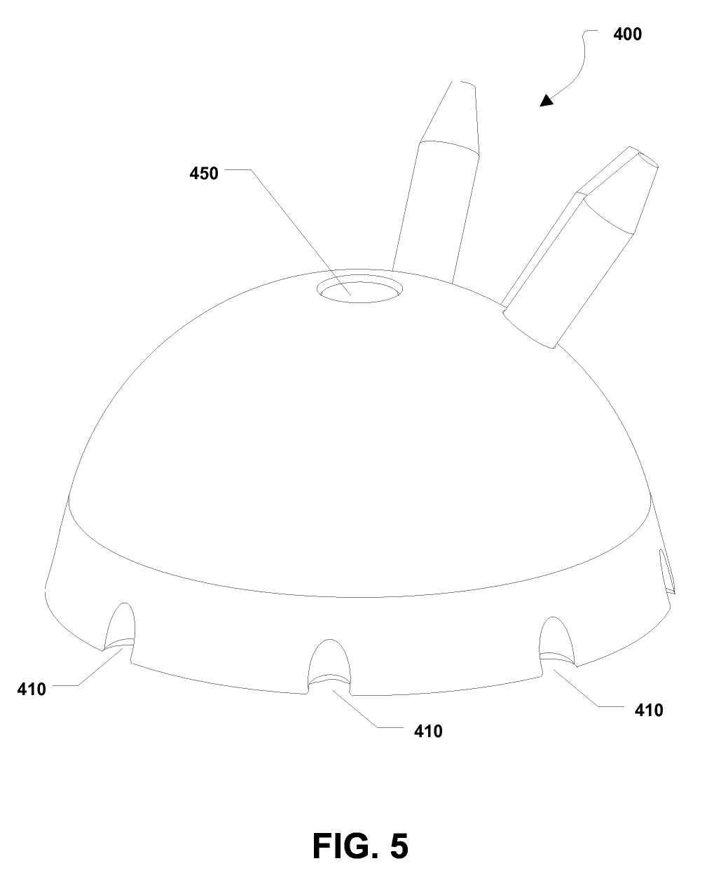

FIG. 4 through FIG. 7 illustrate the details of the acetabular shell 400. As shown, the acetabular shell 400 can include a generally hemi-spherical body 402. The body 402 can include an interior cavity 404. Moreover, the body 402 of the acetabular shell 400 can include an interior surface 406 and an exterior surface 408. The interior surface 406 of the body 402 of the acetabular shell 400 can be concave and the exterior surface 408 of the body 402 of the acetabular shell 400 can be convex.

FIG. 4 indicates that the acetabular shell 400 can also include a plurality of screw engagement holes 410 that can be equally spaced around the perimeter of the acetabular shell 400. Each screw engagement hole 410 can be a semi-cylindrical hole having an inner surface 412. Further, the inner surface 412 of each screw engagement hole 410 can have a central angle 414 that can be greater than one hundred and eighty degrees (180°) and less than three hundred and sixty degrees (360°).

For example, the central angle of each screw engagement hole 410 can be two hundred degrees (200°), two hundred and ten degrees (210°), two hundred and twenty degrees (220°), two hundred and thirty degrees (230°), two hundred and forty degrees (240°), two hundred and fifty degrees (250°), two hundred and sixty degrees (260°), two hundred and seventy degrees (270°), two hundred and eighty degrees (280°), two hundred and ninety degrees (290°), three hundred degrees (300°), three hundred and ten degrees (310°), three hundred and twenty degrees (320°), three hundred and thirty degrees (330°), three hundred and forty degrees (340°), three hundred and fifty degrees (350°), or any other angle between one hundred and eighty degrees (180°) and three hundred and sixty degrees (360°).

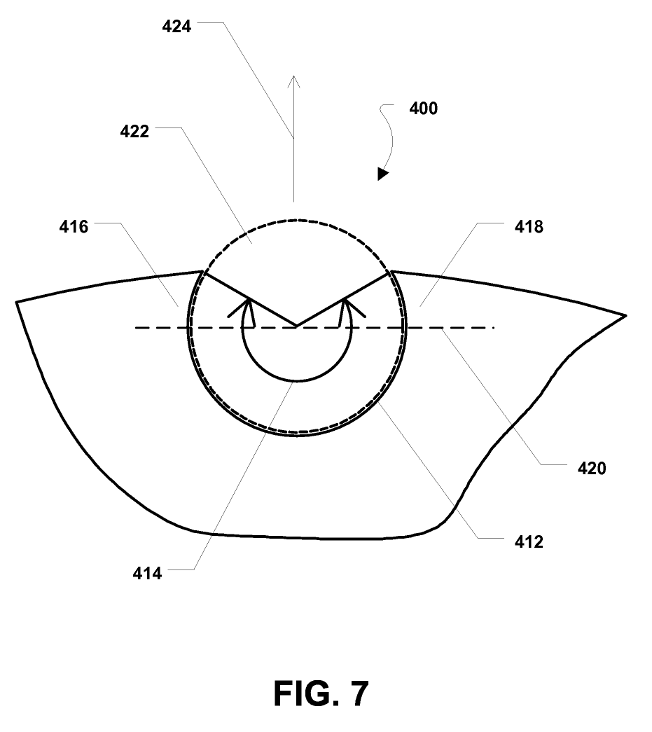

Regardless of the central angle, each screw engagement hole 410 can be flanked by a first screw engagement structure 416 and a second screw engagement structure 418. In a particular embodiment, each screw engagement structure 416, 418 can be a portion of the material surrounding the screw engagement hole 410 between a central axis 420 of the screw engagement hole 410 and an outer perimeter of the acetabular shell 400. Accordingly, the screw engagement portions 416, 418 of each screw engagement hole 410 can partially wrap around a bone screw 422, shown in dashed lines in FIG. 7, and prevent the screw 422 from withdrawing from the screw engagement hole 410 in a radial direction, indicated by arrow 424. In certain embodiments, the screw engagement hole 410 can partially wrap around a head portion of the bone screw 422 or around a circumference of the head portion of the bone screw 422.

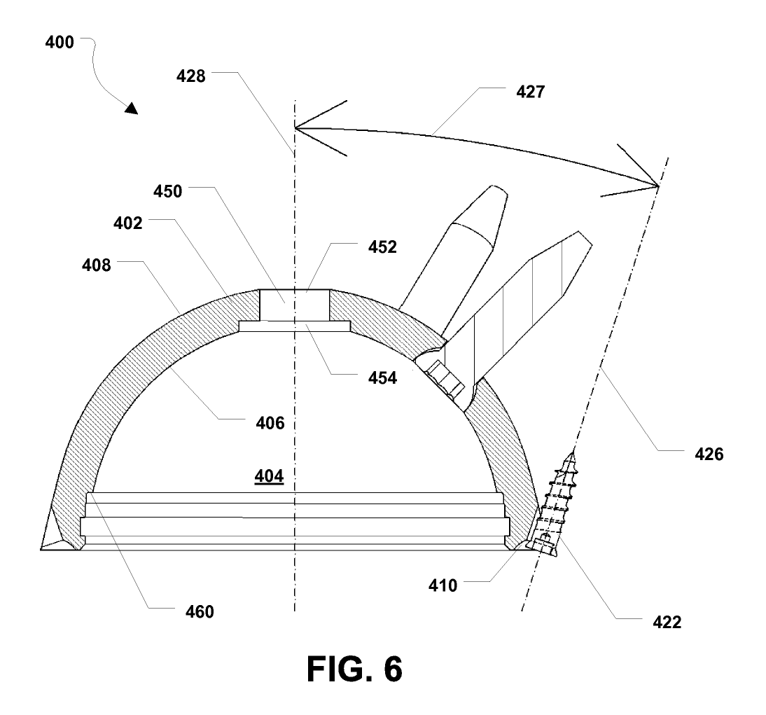

As shown in FIG. 6, the bone screw 422 can be held in place within the acetabular shell 400, e.g., within the screw engagement hole 410 of the acetabular shell 400, such that a longitudinal axis 426 of the bone screw 422 can be at an angle 427 with respect to a longitudinal axis 428 of the acetabular shell 400. In a particular embodiment, the angle 427 can be in a range of ten degrees to twenty-five degrees (10°-25°). Further, the angle 427 can be in a range of fifteen degrees to twenty-one degrees (15°-21°). Also, in a particular embodiment, the angle 427 can be in a range of seventeen degrees to nineteen degrees (17°-19°).

FIG. 4 further shows that the acetabular shell 400 can include a first stabilizing post engagement hole 430, a second stabilizing post engagement hole 432, and a third stabilizing post engagement hole 434. A first stabilizing post 440 can extend through the first stabilizing post engagement hole 430. A second stabilizing post 442 can extend through the second stabilizing post engagement hole 432. Moreover, a third stabilizing post 444 can extend through the third stabilizing post engagement hole 434.

FIG. 4 through FIG. 6 also show that the acetabular shell 400 can include a central hole 450 that can be configured to receive the apex plug 600, described in detail below. In a particular embodiment, the central hole 450 can include a first portion 452 and a relatively larger second portion 454. In a particular embodiment, the first portion 452 can be threaded. As shown, the acetabular shell 400 can also include a seal engagement rim 460. The seal engagement rim 460 can be configured to engage a portion of the seal 500, described in detail below.

In a particular embodiment, the acetabular shell 400 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the acetabular shell 400 can be made from any other substantially rigid materials.

### Description of the Seal

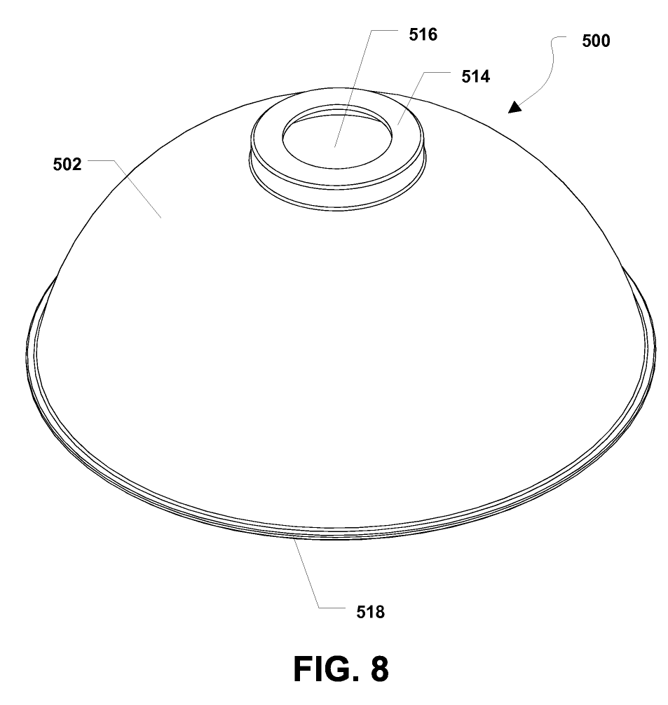

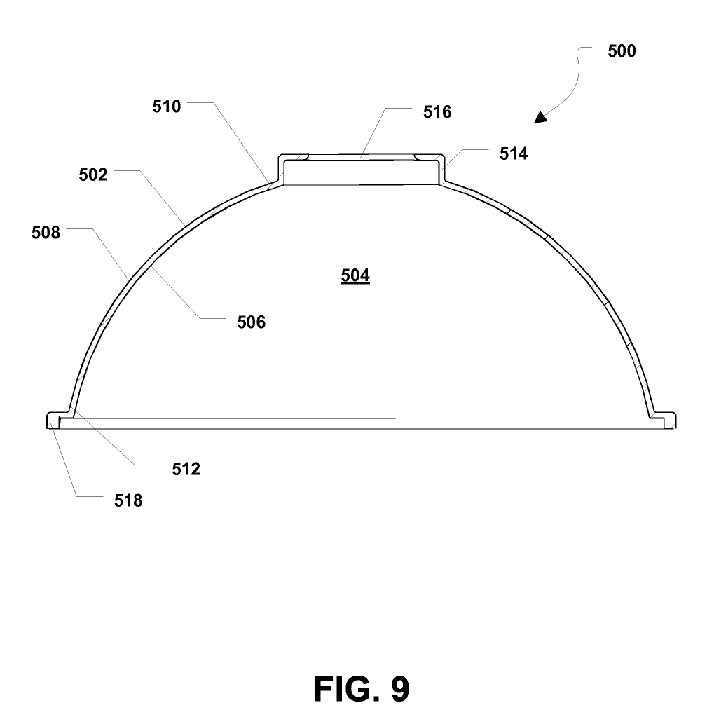

Referring to FIG. 8 and FIG. 9, details concerning the seal 500 are illustrated. As shown, the seal 500 can include a generally hemi-spherical body 502 that can include an interior cavity 504. Further, the body 502 of the seal 500 can include an interior surface 506 and an exterior surface 508. The interior surface 506 can be concave and the exterior surface 508 can be convex. FIG. 8 and FIG. 9 indicate that the body 502 of the seal 500 can include a top 510 and a bottom 512. A central hub 514 can extend from the top 510 of the body 502 of the seal 500. The central hub 514 can include a central hole 516.

As further depicted in FIG. 8 and FIG. 9, the body 502 of the seal 500 can also include an external lip 518 that can extend radially outward from the bottom 512 of the seal 500. As described in greater detail below, the seal 500 can fit into the acetabular shell 400.

In a particular embodiment, the seal 500 can be made from one or more biocompatible, substantially non-bioresorbable materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the seal 500 can be made from another biocompatible material. In an alternative embodiment, the seal 500 can be made sprayed into the acetabular shell 400 and cured in place.

### Description of the Apex Plug

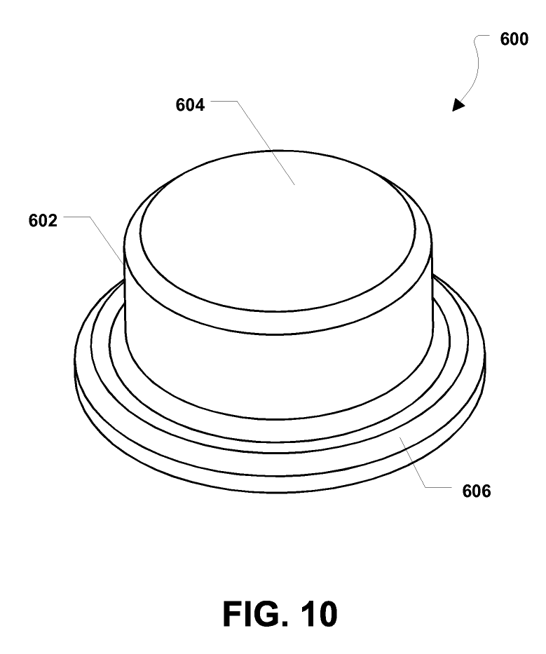

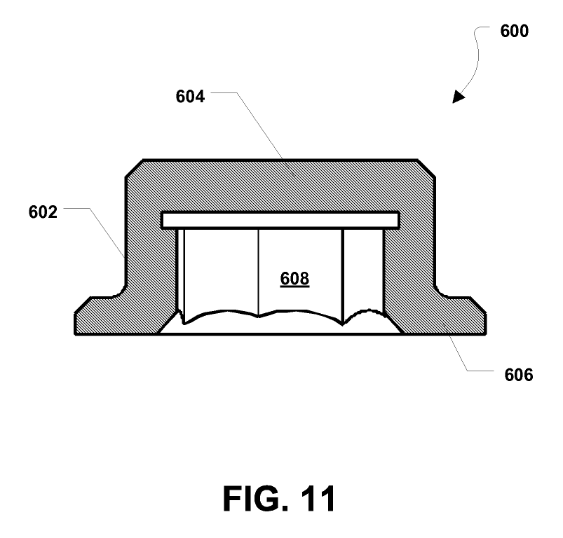

FIG. 10 and FIG. 11 illustrate the details concerning the construction of the apex plug 600. As shown, the apex plug 600 can include a body 602 that can have a first portion 604 and a second portion 606 that can be relatively larger than the first portion 604. Further, the apex plug 600 can include an interior cavity 608 that can be sized and shaped to receive an end of a tool, e.g., an Allen wrench. Alternatively, the interior cavity 608 can be sized and shaped to receive a slotted screwdriver, a Phillips screwdriver, a Torx screwdriver, or any other screwdriver well known in the art.

In a particular embodiment, the first portion 604 of the apex plug 600 can be sized and shaped to fit into the first portion 452 of the central hole 450 formed in the acetabular shell 400. Further, in a particular embodiment, the first portion 604 of the apex plug 600 can be threaded and the first portion 604 of the apex plug 600 can be threadably engaged with the first portion 452 of the central hole 450 in the acetabular shell 400.

In a particular embodiment, the apex plug 600 can be made from one or more rigid materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the apex plug 600 can be made from any other substantially rigid materials.

### Description of the Liner

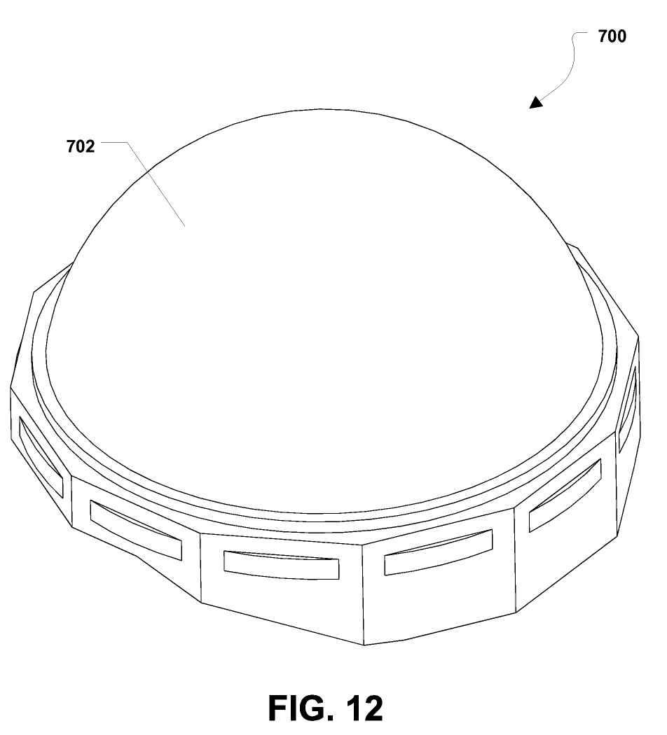

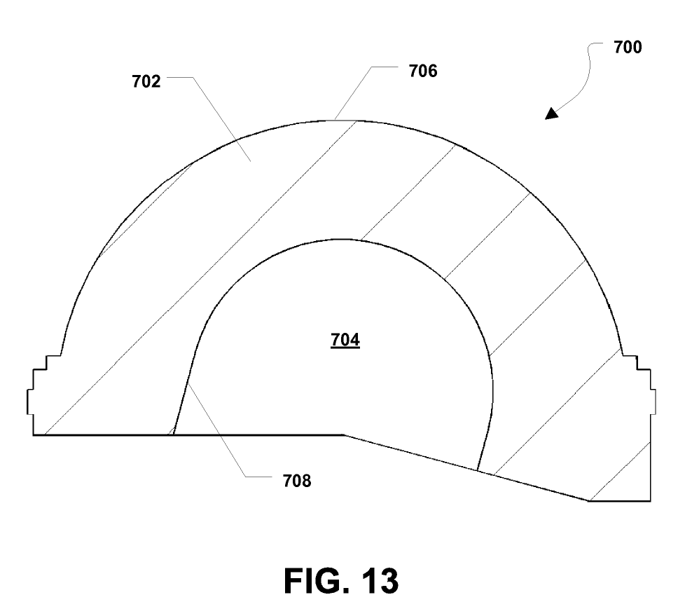

Referring to FIG. 12 and FIG. 13, the liner 700 is illustrated. As depicted, the liner 700 can include a generally hemi-spherical body 702 that can include an interior cavity 704. Further, the liner 700 can include a generally convex exterior surface 706 and a generally concave interior surface 708. In a particular embodiment, the interior cavity 704 of the liner 700 can be sized and shaped to receive a femoral head of a femoral implant. In a particular embodiment, the liner 700 can be made from ultra-high molecular weight polyethylene (UHMWP). Alternatively, the liner 700 can be made from another biocompatible material.

### Description of the Assembled Acetabular Implant

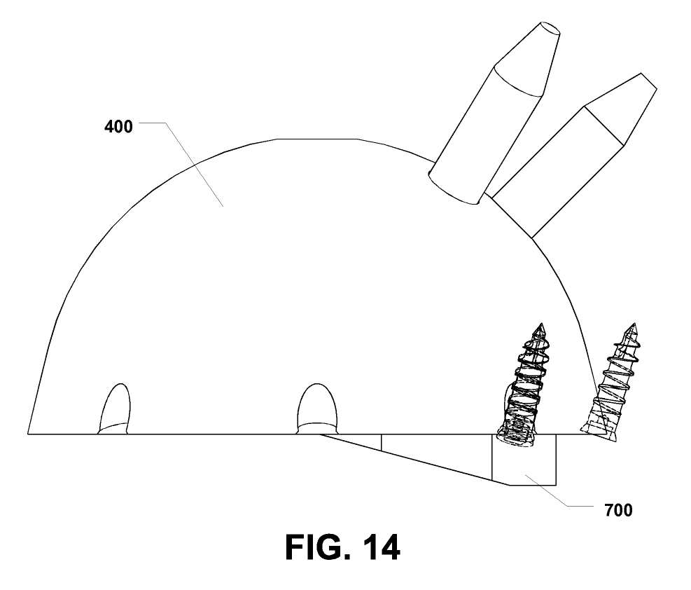

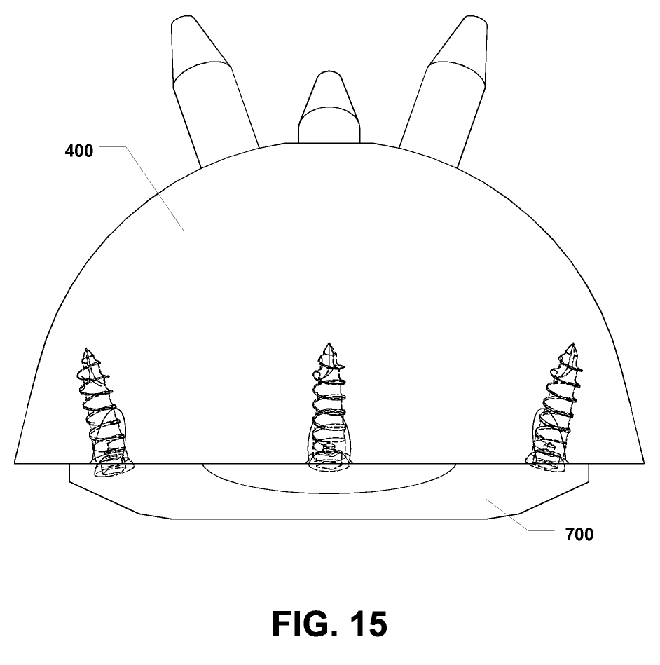

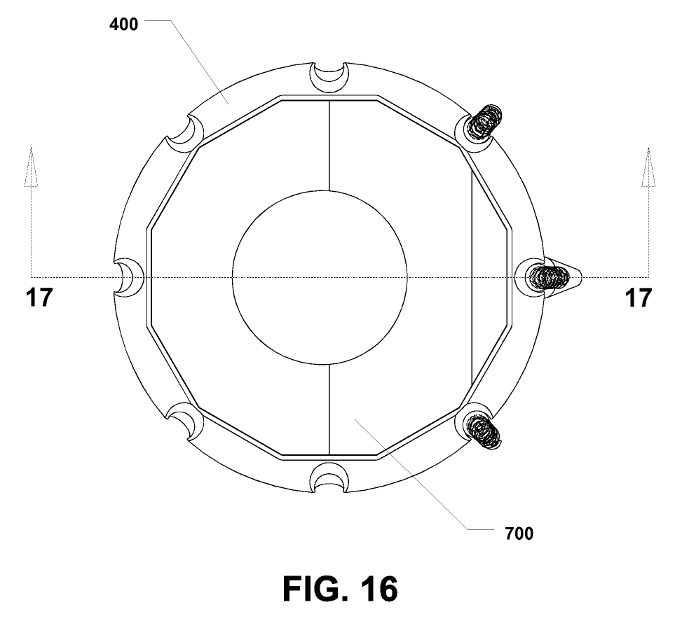

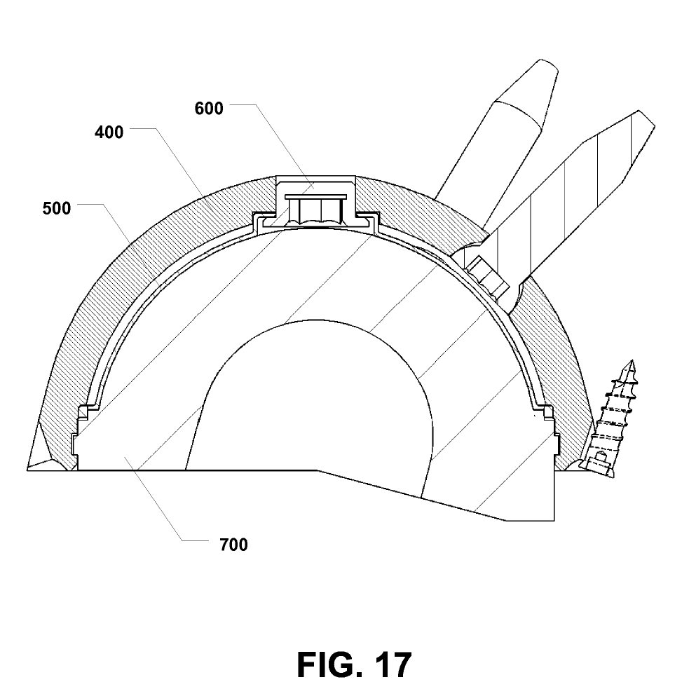

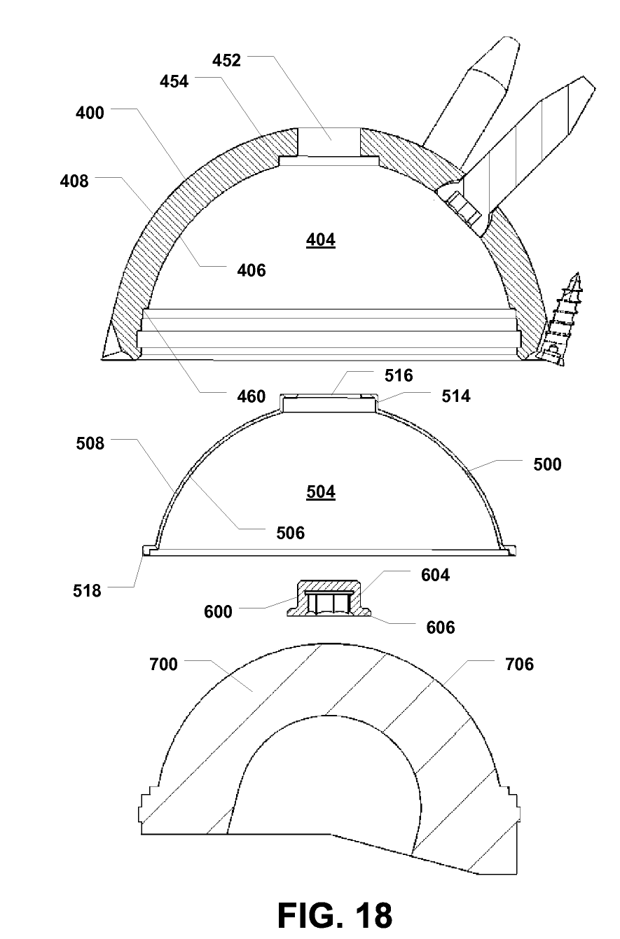

FIG. 14 through FIG. 18 show the acetabular implant 300 assembled. As shown in FIG. 17 and FIG. 18, when the acetabular implant 300 is assembled, the seal 500 can be placed within the interior cavity 404 of the acetabular shell 400. Also, the liner 700 can be placed within the interior cavity 504 of the seal 500. In particular, the exterior surface 508 of the seal 500 can be adjacent to the interior surface 406 of the acetabular shell 400. Further, the exterior surface 706 of the liner 700 can be adjacent to the interior surface 506 of the seal 500.

As shown in FIG. 17 and FIG. 18, the central hub 514 of the seal 500 can fit into the central hole 450 formed in the acetabular shell 400. In a particular embodiment, the central hub 514 of the seal 500 can fit into the second portion 454 of the central hole 450 of the acetabular shell 400. Further, the apex plug 600 can be inserted through the central hub 514 of the seal 500, e.g., through the central hole 516 formed therein, and engaged with the central hole 450 in the acetabular shell 400. In a particular embodiment, the first portion 604 of the apex plug 600 can be engaged with the first portion 452 of the central hole 450 in the acetabular shell 400. For example, the first portion 604 of the apex plug 600 can be threadably engaged with the first portion 452 of the central hole 450 in the acetabular shell 400. Alternatively, the first portion 604 of the apex plug 600 can be press fit into the first portion 452 of the central hole 450 within the acetabular shell 400.

As shown in FIG. 17 and FIG. 18, the second portion 606 of the apex plug 600 can bind the central hub 514 of the seal 500 within the second portion 454 of the central hole 450 in the acetabular shell 400. In other words the central hub 514 of the seal 500 can be sandwiched between the second portion 606 of the apex plug 600 and the acetabular shell 400. The apex plug 600 can maintain the seal 500 within the acetabular shell 400 and prevent the seal 500 from being expelled from the acetabular shell 400. Further, the external lip 518 of the seal 500 can engage the seal engagement rim 460 of the acetabular shell 400. Accordingly, the seal 500 can substantially prevent any debris from the liner 700 from migrating through the acetabular shell 400.

### Description of a Method of Treating Hip Joint

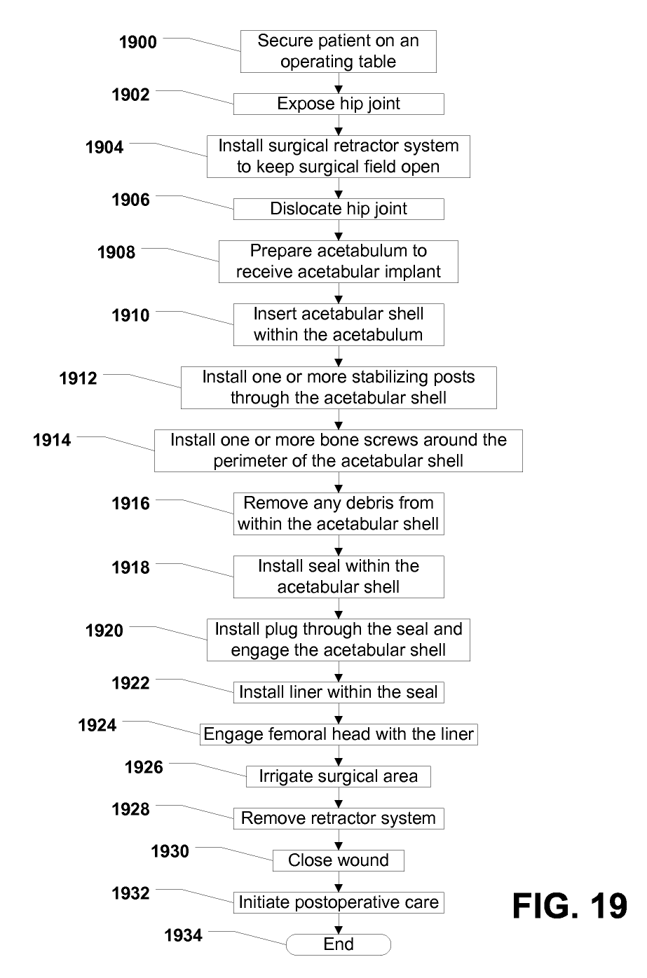

Referring to FIG. 19, a method of treating a hip joint is illustrated and commences at block 1900. At block 1900, a patient can be secured on an operating table. For example, the patient can be secured on the operating table in a supine position. At block 1902, a hip joint of the patient can be exposed. The hip joint can be exposed by making an incision at the patient's lateral side adjacent to the hip joint through the skin and muscle. At block 1904, a surgical retractor system can be installed in order to keep the surgical field open.

Moving to block 1906, the hip joint can be dislocated. In other words, a femoral head can be removed from an acetabulum in order to completely expose the acetabulum and the femoral head. At block 1908, the acetabulum can be prepared to receive an acetabular implant, e.g., the acetabular implant described herein. The acetabulum can be prepared by removing bone in or around the acetabulum. Proceeding to block 1910, the acetabular shell can be inserted into the acetabulum. Further, at block 1912, one or more stabilizing rods can be installed through the acetabular shell. In a particular embodiment, the one or more stabilizing rods can engage bone in or around the acetabulum.

Continuing to block 1914, one or more bone screws can be installed around the perimeter of the acetabular shell. In particular, each bone screw can be installed through a screw engagement hole and can be engaged with bone around the acetabulum. At block 1916, any debris within the acetabular shell can be removed. Thereafter, at block 1918, a seal can be installed within the acetabular shell. Moreover, at block 1920, an apex plug can be installed through the seal and can be engaged with the acetabular shell. At block 1922, a liner can be installed within the seal.

Proceeding to block 1924, a femoral head can be engaged within the liner. For example, the femoral head can be installed within an interior cavity formed in the liner. The femoral head can be a natural femoral head or an artificial femoral head. At block 1926, the surgical area can be irrigated. Also, at block 1928, the retractor system can be removed. Further, at block 1930, the surgical wound can be closed. The surgical wound can be closed by simply allowing the patient's skin to close due to the elasticity of the skin. Alternatively, the surgical wound can be closed using sutures, surgical staples, or any other suitable surgical technique well known in the art. At block 1932, postoperative care can be initiated. The method can end at state 1934.

### CONCLUSION

With the configuration of structure described above, the acetabular implant provides a device that can be used to treat a hip joint. For example, the acetabular implant can be installed within an acetabulum of a pelvis. Further, the seal within the acetabular implant can prevent debris from an UHMWPE liner within the acetabular implant from migrating through an acetabular shell in which the liner is installed. As such, the acetabular implant can substantially reduce osteolytic lesions around the acetabular implant due to UHMWPE debris. Additionally, the seal can substantially prevent bone screws inserted through the acetabular shell and into bone from backing out of the bone. The seal can also be used in conjunction with other implants designed to replace ball-and-socket joints. For example, the seal can be used in a shoulder implant having a first component, e.g., a cup-shaped component, designed to fit into a glenoid of a scapula, and a second component, e.g., a head, designed to fit into the first cup-shaped component. In such an application, the seal can substantially prevent debris from the second component from migrating through the first cup-shaped component.

Further, the shape of the acetabular shell, e.g., the shape of the screw engagement holes formed in the acetabular shell results in a relatively lower overall diameter of the acetabular implant. In other words, since each screw engagement hole does not completely surround a bone screw, but extends around the shoulders of the bone screw, extra material that would otherwise increase the overall diameter of the acetabular implant can be eliminated. The configuration of the screw engagement hole described herein can be incorporated in any implant device that can be fixed in place using one or more screws, e.g., an acetabular shell, a tibial base, a trauma plate, a cervical plate, a glenoid cup, etc.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An acetabular implant, comprising:
an acetabular shell;
a liner installed within the acetabular shell; and
a seal installed between the liner and the acetabular shell, wherein the seal substantially prevents debris from the liner from migrating through the acetabular shell.

2. The acetabular implant of claim 1, wherein the acetabular shell comprises:
a generally hemi-spherical shell body;
an interior cavity within the shell body; and
a central hole extending from the interior cavity through the shell body.

3. The acetabular implant of claim 2, wherein the seal comprises:
a generally hemi-spherical seal body sized and shaped to fit within the shell body, wherein the seal body defines a top and a bottom;
a hub extending from the top of the seal body, wherein the hub is sized and shaped to fit within the central hole of the shell body; and
a central hole formed in the hub.

4. The acetabular implant of claim 3, wherein the central hole of the shell body includes a first portion and a second portion, wherein a diameter of the second portion is larger than a diameter of the first portion.

5. The acetabular implant of claim 4, wherein the hub of the seal body is sized and shaped to fit within the second portion of the central hole in the shell body.

6. The acetabular implant of claim 5, further comprising:
an apex plug comprising an apex plug body having a first portion and a second portion, wherein:
a diameter of the second portion is larger than a diameter of the first portion;
the first portion of the apex plug body is sized and shaped to fit through the central hole in the hub of the seal body;
the first portion of the apex plug is sized and shaped to fit into and engage the first portion of the central hole in the shell body; and
the second portion of the apex plug is sized and shaped to bind the hub of the seal body in the second portion of the central hole in the shell body.

7. The acetabular implant of claim 3, wherein the acetabular shell further comprises:
a seal engagement rim formed within the interior cavity of the shell body.

8. The acetabular implant of claim 7, wherein the seal further comprises:
an external lip extending radially outward from the bottom of the seal body, wherein the external lip of the seal body is sized and shaped to engage the seal engagement rim within the interior cavity of the shell body.

9. The acetabular implant of claim 8, wherein the seal further comprises:
an interior cavity within the seal body.

10. The acetabular implant of claim 9, wherein the liner comprises:
a generally hemi-spherical liner body sized and shaped to fit into the interior cavity of the seal body; and
an interior cavity sized and shaped to receive a femoral head.

11. The acetabular implant of claim 2, wherein the acetabular shell further comprises:
a plurality of screw engagement holes around the perimeter of the shell body, wherein each screw engagement hole is a semi-cylindrical hole having an inner surface.

12. The acetabular implant of claim 11, wherein the inner surface of each screw engagement hole includes a central angle greater than one hundred and eighty degrees (180°) and less than three hundred and sixty degrees (360°).

13. The acetabular implant of claim 12, wherein the acetabular shell further comprises a first screw engagement structure and a second screw engagement structure adjacent to each screw engagement hole.

14. The acetabular implant of claim 13, wherein each screw engagement structure comprises a portion of the acetabular shell between a central axis of the screw engagement hole and an outer perimeter of the acetabular shell.

15. The acetabular implant of claim 14, wherein each screw engagement structure is configured to at least partially wrap around a bone screw disposed within the screw engagement hole and substantially prevent the screw from withdrawing from the screw engagement hole in a radial direction relative to the acetabular shell.

16. The acetabular implant of claim 11, further comprising a bone screw engaged with each screw engagement hole.

17. The acetabular implant of claim 16, wherein each bone screw is held in place within the acetabular shell so that a longitudinal axis of the bone screw is at an angle with respect to a longitudinal axis of the acetabular shell.

18. The acetabular implant of claim 17, wherein the angle is in a range of ten degrees to twenty-five degrees (10°-25°).

19. The acetabular implant of claim 18, wherein the angle is in a range of fifteen degrees to twenty-one degrees (15°-21°).

20. The acetabular implant of claim 19, wherein the angle is in a range of seventeen degrees to nineteen degrees (17°-19°).

21. A seal for an acetabular implant, comprising:
a generally hemi-spherical seal body, wherein the seal body is configured to be placed between an acetabular shell and a liner and wherein the seal body can substantially prevent debris from the liner from migrating through the acetabular shell.

22. The seal of claim 21, wherein the seal body further comprises:
a top;
a bottom; and
a hub extending from the top of the seal body, wherein the hub is sized and shaped to fit within a central hole in the acetabular shell.

23. The seal of claim 22, wherein the seal body further comprises:
a central hole formed in the hub, wherein the central hole is configured to receive an apex plug therethrough.

24. The seal of claim 23, wherein the seal body further comprises:
an external lip extending radially outward from the bottom of the seal body, wherein the external lip of the seal body is sized and shaped to engage a seal engagement rim within the acetabular shell.

25. The seal of claim 24, wherein the seal body further comprises:
an interior cavity wherein the interior cavity is sized and shaped to receive the liner.

26. A method of treating a hip joint, comprising:
exposing the hip joint;
preparing an acetabulum to receive an acetabular implant;
installing an acetabular shell within the acetabulum; and
installing a seal within the acetabular shell.

27. The method of claim 26, further comprising:
installing an apex plug through the seal; and
engaging the apex plug with the acetabular shell, wherein the apex plug maintains the seal within the acetabular shell.

28. The method of claim 27, further comprising:
installing a liner within the seal.

29. The method of claim 26, further comprising:
installing one or more stabilizing posts through the acetabular shell before installing the seal.

30. An implant, comprising:
a first component;
a second component configured to be installed in the first component; and
a seal configured to be installed between the first component and the second component, wherein the seal substantially prevents debris from the second component from migrating through the first component.

31. A seal for an orthopedic implant, comprising:
a generally hemi-spherical seal body, wherein the seal body is configured to be placed between a first component and a second component and wherein the seal body substantially prevents debris from the second component from migrating through the first component.
